(19) United States
(12) Patent Application Publication
(10) Pub. No.: US 2018/0185434 A1
(43) Pub. Date: Jul. 5, 2018

(54) IMMUNE CHECKPOINT CHIMERIC ANTIGEN RECEPTORS THERAPY

(71) Applicant: The Johns Hopkins University, Baltimore, MD (US)

(72) Inventors: Ivan M. Borrello, Baltimore, MD (US); Susan Lee, Baltimore, MD (US); Kimberly A. Noonan, Baltimore, MD (US); Drew M. Pardoll, Brookeville, MD (US)

(21) Appl. No.: 15/740,981

(22) PCT Filed: Jun. 29, 2016
(86) PCT No.: PCT/US16/40010

§ 371 Date: Dec. 29, 2017

Related U.S. Application Data
(60) Provisional application No. 62/186,108, filed on Jun. 29, 2015.

(51) Int. Cl. A61K 38/00; A61K 38/17; C07K 14/725; C07K 14/705; C07K 19/00; C12N 15/62; A61K 35/17; A61K 48/00; A61P 35/02; C12N 15/82

(52) U.S. Cl. CPC A61K 38/005; A61K 38/1709; C07K 14/7051; C07K 14/70521; C07K 14/70596; C12N 15/8201; C12N 15/62; A61K 35/17; A61K 48/005; A61P 35/02; C07K 19/00

(57) ABSTRACT

In some aspects, the embodiments relate to compositions and methods related to chimeric transmembrane proteins. The chimeric transmembrane proteins may comprise the extracellular domain of an inhibitory receptor, and an intracellular signaling domain that can activate an immune response.

### CROSS-REFERENCE TO RELATED APPLICATIONS

This application claims priority to U.S. Provisional Application No. 62/186,108, filed Jun. 29, 2015, which is hereby incorporated by reference in its entirety.

### BACKGROUND

The large majority of patients with malignancies will die from their disease. One approach to treating these patients is to genetically modify T cells to target antigens expressed on tumor cells through the expression of chimeric antigen receptors (CARs). CARs are antigen receptors that are designed to recognize cell surface antigens in a human leukocyte antigen-independent manner. Outside of the successes with CD19-targeted approaches, attempts at using genetically modified cells expressing CARs to treat other malignancies have met with limited success.

Recently, checkpoint inhibiting antibodies targeting CTLA-4 (ipilimumab) and PD-1 (nivolumab, pembrolizumab) have shown considerable activity in the treatment of various malignancies including metastatic melanoma, non small cell lung cancer (NSCLC) and Hodgkin's lymphoma. These data demonstrate how checkpoint blockade represents a major obstacle to effective immunotherapy by overcoming T cell anergy.

### SUMMARY

In some aspects, the embodiments relate to a chimeric transmembrane protein, comprising the extracellular domain of an inhibitory receptor and an intracellular signaling domain that can activate an immune response. The extracellular domain may be, for example, an extracellular domain from CTLA-4, PD-1, LAG-3, or Tim-3. The intracellular signaling domain may be, for example, the intracellular signaling domain of CD3ζ, 4-1BB, or CD28. In some aspects, the embodiments relate to a nucleic acid encoding a chimeric transmembrane protein as described herein.

In some aspects, the embodiments relate to cells, comprising a nucleic acid encoding a chimeric transmembrane protein as described herein. In some aspects, the embodiments relates to cells, comprising a chimeric transmembrane protein as described herein.

In some aspects, the embodiments relate to methods for making recombinant cells, comprising transfecting cells with a nucleic acid encoding a chimeric transmembrane protein as described herein.

In some aspects, the embodiments relate to methods for increasing an immune response in a subject, comprising administering to the subject a recombinant cell as described herein. In some aspects, the embodiments relate to methods for treating a neoplasm in a subject, comprising administering to the subject a recombinant cell as described herein.

### BRIEF DESCRIPTION OF THE DRAWINGS

FIG. 1 shows a nucleotide sequence (SEQ ID NO:1) encoding a chimeric transmembrane protein, comprising a leader peptide from CD8 (“CD8a LP”), the extracellular domain of mouse PD-1 (“PD-1 ECD”), and the transmembrane and intracellular domains of mouse 4-1BB (“4-1BB TM” and “4-1BB ICD”, respectively). The reverse complement of the nucleotide sequence (SEQ ID NO:2) is also shown. Codons were optimized for expression in mouse lymphocytes.

FIG. 2 shows a nucleotide sequence (SEQ ID NO:3) encoding a chimeric transmembrane protein, comprising a leader peptide from CD8 (“CD8a LP”), the extracellular domain of human PD-1 (“PD-1 ECD”), and the transmembrane and intracellular domains of human 4-1BB (“4-1BB TM” and “4-1BB ICD”, respectively). The reverse complement of the nucleotide sequence (SEQ ID NO:4) is also shown. Codons were optimized for expression in human lymphocytes.

FIG. 3 shows flow cytometry results for Lenti-X 293T cells transfected with a mCherry gene and a nucleic acid encoding a chimeric transmembrane protein (SEQ ID NO:1), comprising the extracellular domain of PD-1 using the transfection protocol described in Example 2, infra. FIG. 3 shows that the nucleic acid is expressed in 293T cells.

FIG. 4 shows flow cytometry results for Lenti-X 293T cells transduced with a mCherry gene and a nucleic acid encoding a chimeric transmembrane protein (SEQ ID NO:1), comprising the extracellular domain of PD-1 and the intracellular domain of 4-1BB, using the transduction protocol described in Example 1, infra. Cells were transduced in 1 well of a 6-well plate with 1.9 mL of virus. FIG. 4 shows that the nucleic acid is expressed in 293T cells.

FIG. 5 shows flow cytometry results for Lenti-X 293T cells transduced with a mCherry gene and a nucleic acid encoding a chimeric transmembrane protein (SEQ ID NO:1), comprising the extracellular domain of PD-1 and the intracellular domain of 4-1BB, using the transduction protocol described in Example 1, infra. Cells were transduced in 1 well of a 6-well plate with 0.38 mL of virus. FIG. 5 shows that the nucleic acid is expressed in 293T cells.

FIG. 6, Panel A and Panel B illustrates that MILs comprising a chimeric receptor having a PD-1 extracellular domain, a 4-1BB transmembrane domain, and a 4-1BB intracellular domain do not negatively affect tumor specificity.

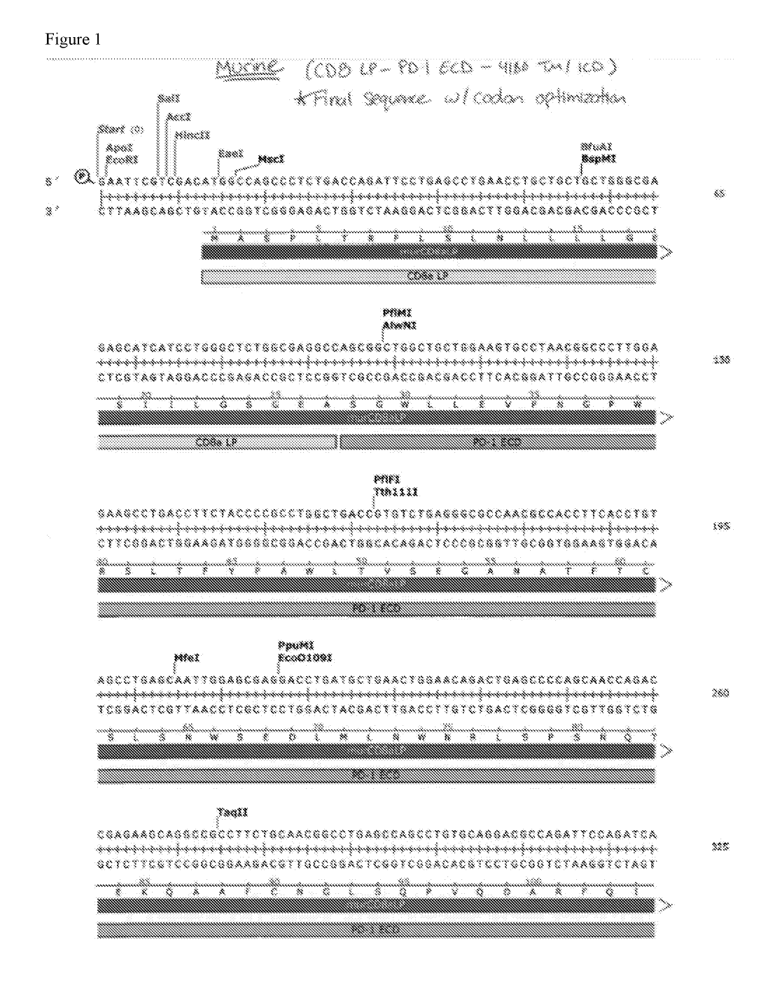

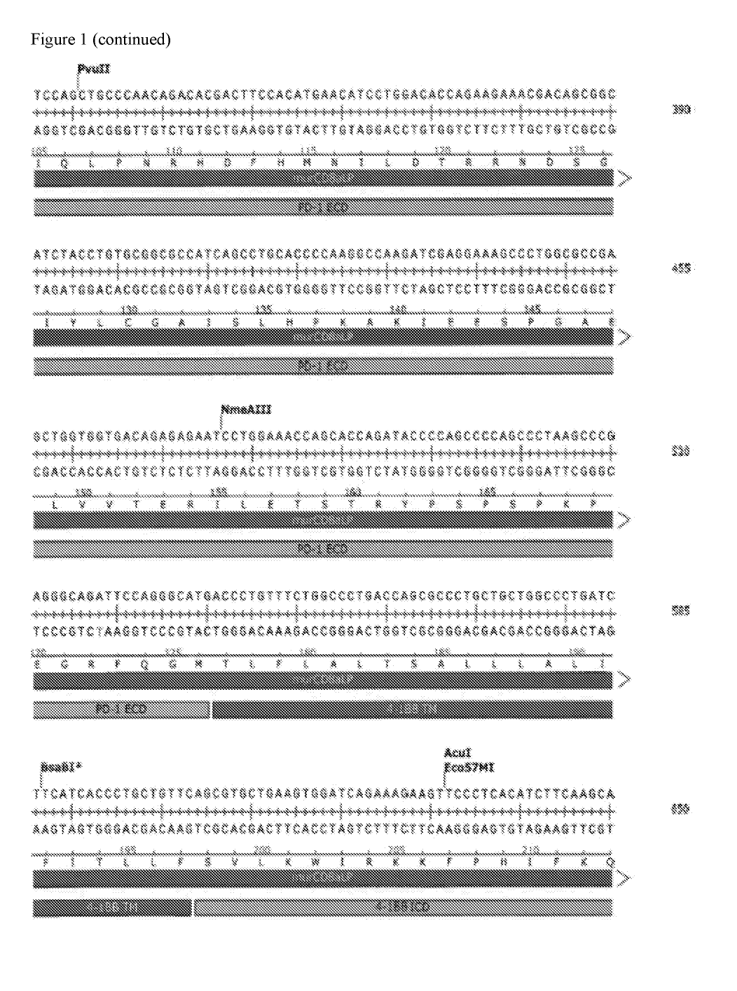

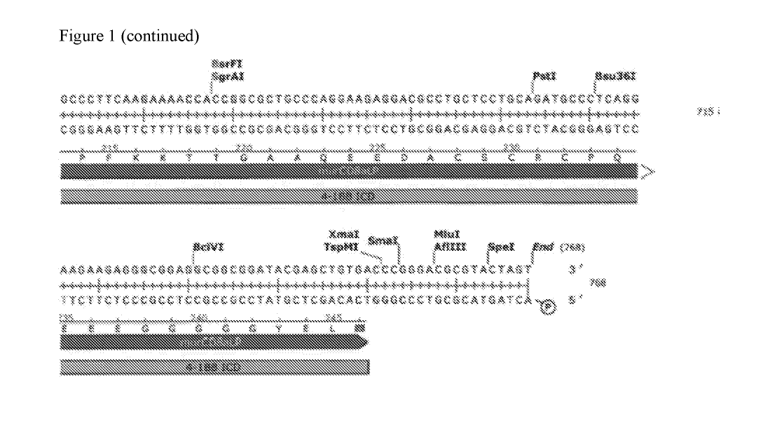

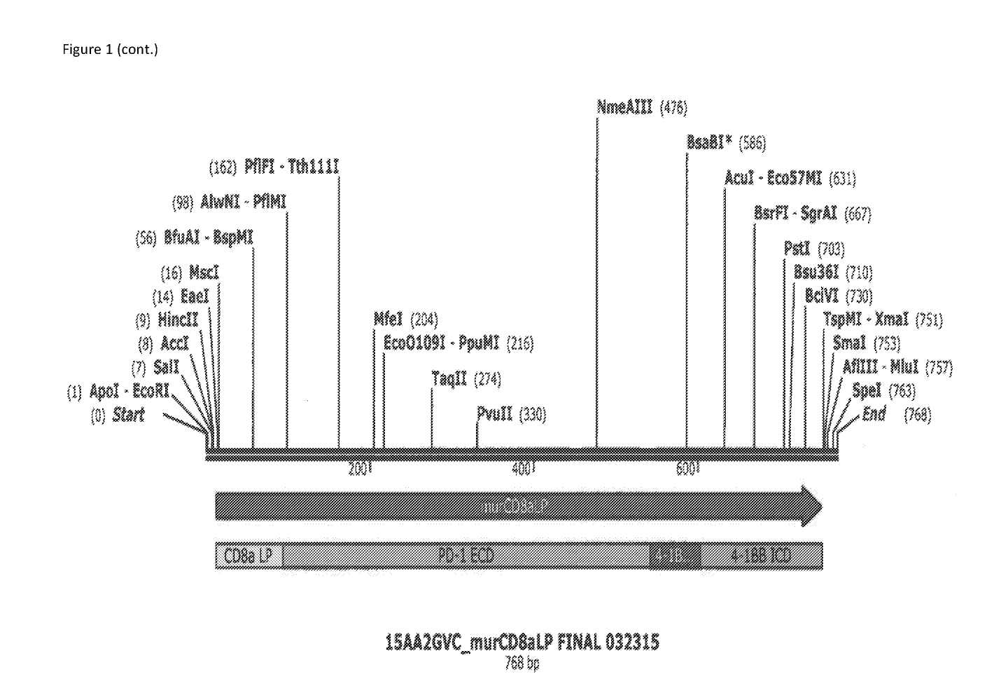

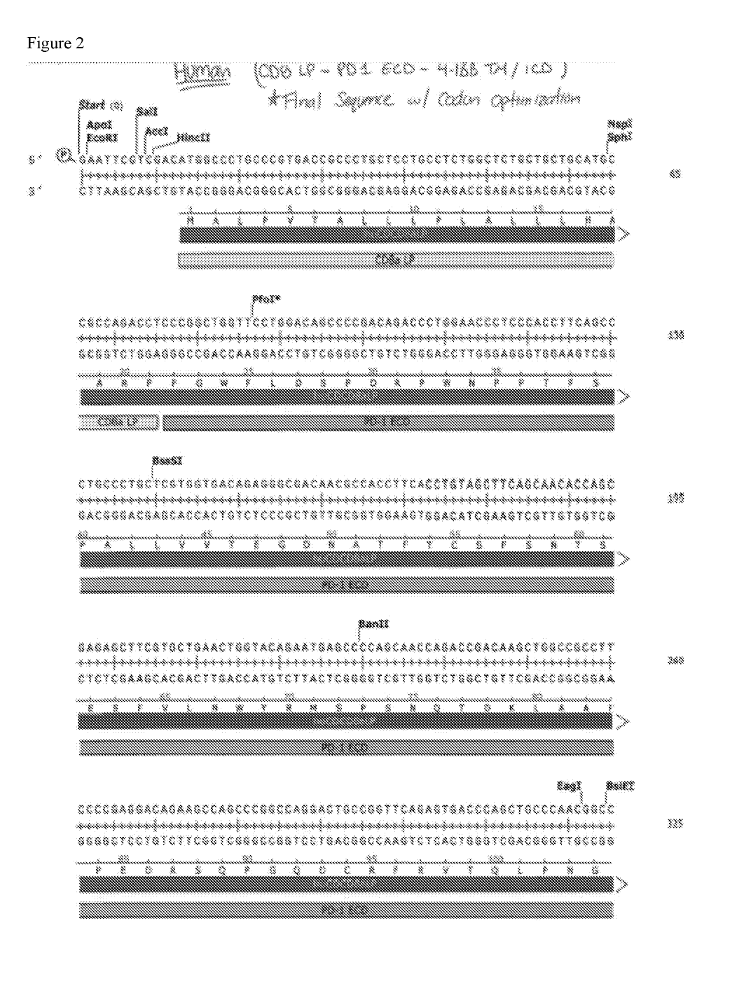

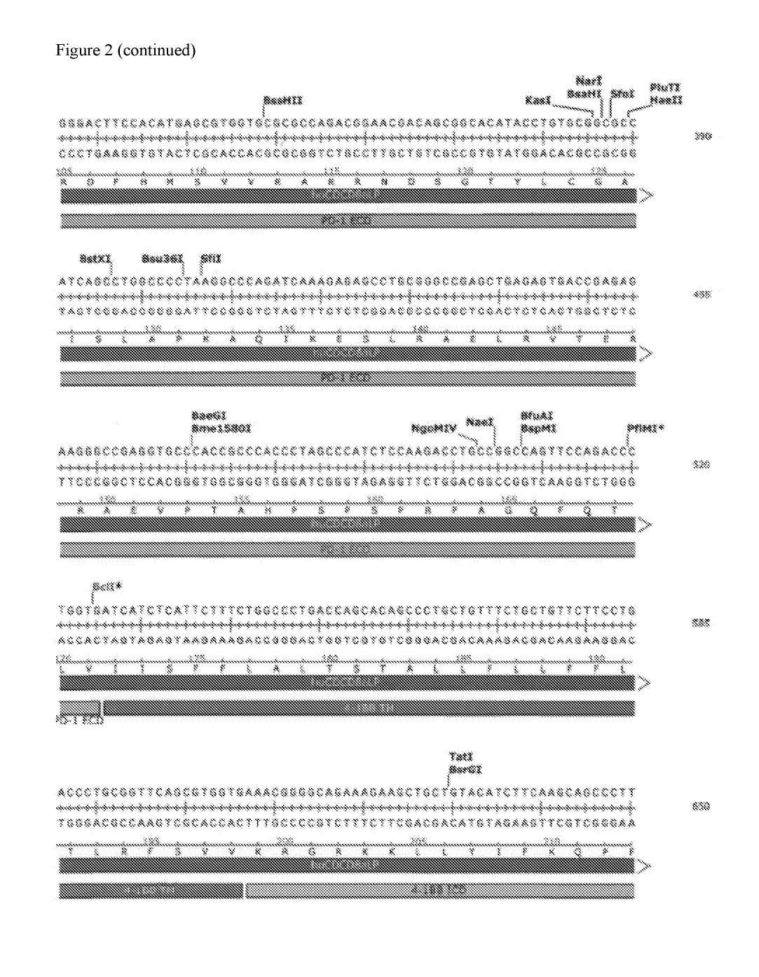

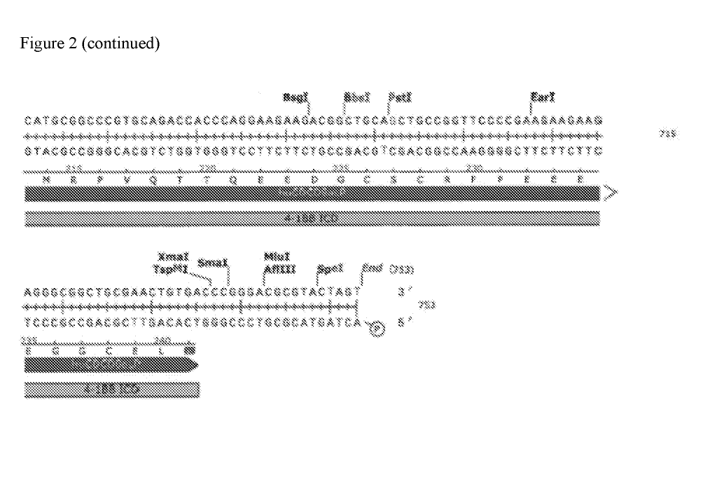

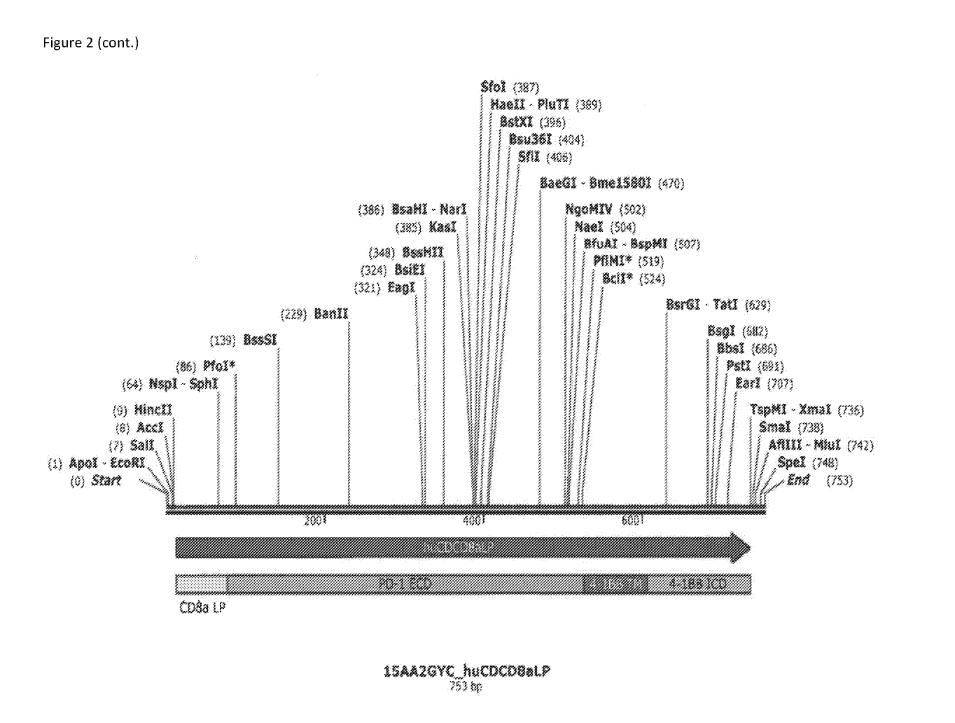

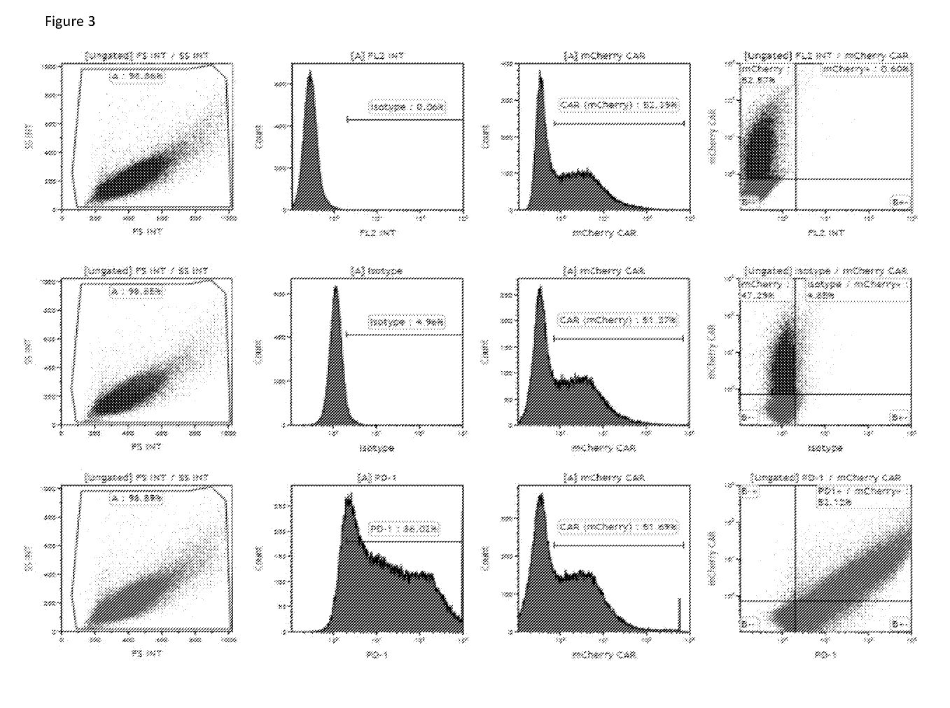

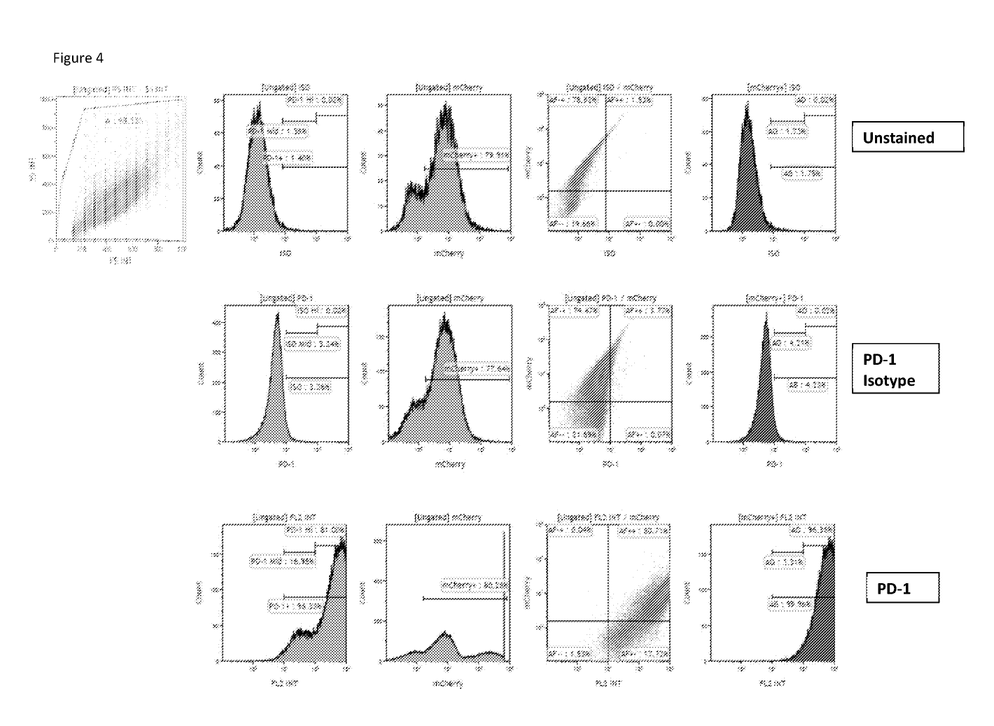

### DETAILED DESCRIPTION

CAR therapy has shown significant promise to date. CD19 CARs targeting chronic lymphocytic leukemia (CLL) and more recently, acute lymphoblastic leukemia (ALL) have met with notable success. Interestingly, CARs targeting other antigens have not provided similar clinical responses. One limitation of such antigen-targeted approaches is their therapeutic applicability, which is limited only to the diseases expressing particular surface receptors and the limitations of targeting a single tumor antigen that have resulted in relapses with antigen-loss variants.

A major hurdle in tumor immunology is the induction of tumor-specific tolerance which limits the intrinsic anti-tumor efficacy of many cell based approaches. Recent studies have shown significant clinical efficacy by targeting checkpoint inhibitors leading to the approval of anti-CTLA-4 and anti-PD-1 for metastatic melanoma. In some aspects, the embodiments relate to a chimeric receptor, comprising an extracellular domain expressing of a checkpoint inhibitor and an activating intracellular domain. This has the advantage of hijacking the tolerogenic mechanisms into activating signals. This approach can be used in all clinical situations in which T cell anergy is a major aspect of the pathogenesis of the disease and where the antigen specificity is provided by the endogenous T cell repertoire.

In some aspects, the embodiments relate to a chimeric transmembrane protein, comprising an extracellular domain of an inhibitory receptor, a transmembrane domain, and an intracellular signaling domain. In some embodiments, the intracellular signaling domain can activate an immune response. The intracellular signaling domain may comprise a portion of an intracellular signaling protein. In some embodiments, the intracellular domain can be used to maintain the activation of a cell, such as a T-cell.

In some embodiments, the extracellular domain can transduce a signal to the intracellular signaling domain. For example, the extracellular domain may transduce a signal to the intracellular signaling domain upon binding an agonist of the native inhibitory receptor.

Signal transduction may comprise oligomerization of the protein. Oligomerization may comprise homo-oligomerization or hetero-oligomerization. Oligomerization may comprise dimerization of the protein, i.e., homo-dimerization with a second chimeric transmembrane protein or hetero-dimerization with a different protein.

Signal transduction may comprise phosphorylation. For example, the intracellular signaling domain may comprise kinase activity and/or a phosphorylation site. Signal transduction may comprise autophosphorylation, e.g., autophosphorylation of the intracellular signaling domain.

In some embodiments, the protein comprises a transmembrane domain. In some embodiments, the protein is an integral membrane protein. For example, the protein may be a type 1 membrane protein, a type 2 membrane protein, or a multi-spanning membrane protein. In some embodiments, the protein comprises the transmembrane domain of the inhibitory receptor. In some embodiments, the protein comprises the transmembrane domain of the intracellular signaling protein. The chimeric transmembrane protein may comprise a signal peptide, e.g., to translocate the extracellular domain across a cell membrane. In some embodiments, the transmembrane domain comprises the sequence of IISFFLALTSTALLFLLFFLTLRFSVV (SEQ ID NO: 5). In some embodiments, the chimeric transmembrane protein comprises a signal peptide derived from CD8. In some embodiments, the signal peptide comprises the CD8 leader peptide. In some embodiments, the signal peptide comprises MALPVTALLLPLALLLHAARP (SEQ ID NO: 6).

In some embodiments, the extracellular domain is the extracellular domain of an inhibitory receptor. In some embodiments, the extracellular domain comprises a ligand-binding domain, e.g., the agonist-binding domain of the inhibitory receptor. In some embodiments, the extracellular domain comprises sufficient structure to transduce a signal across the membrane in response to ligand binding. Without being bound to any particular theory, for inhibitory receptors that transduce a signal by oligomerization mediated by a multivalent ligand, the mere presence of a ligand-binding domain may be sufficient structure to transduce a signal across the membrane in response to ligand binding. Without being bound to any particular theory, for inhibitory receptors that transduce a signal by altering the orientation of a transmembrane domain relative to the cell membrane, the extracellular domain may require native structure between the ligand-binding domain and transmembrane domain to transduce a signal across the membrane in response to ligand binding. For example, an extracellular domain may comprise the native sequence of the inhibitory receptor from its ligand-binding domain to its transmembrane domain.

The native inhibitory receptor can be a human inhibitory receptor or a mouse inhibitory receptor. Thus, the extracellular domain may comprise a human or mouse amino acid sequence. In some embodiments, the origin of the native inhibitory receptor is selected to match the species of a subject that is being treated, e.g., to avoid an immune response against the chimeric transmembrane protein. Nevertheless, the native inhibitory receptor may be selected from a different species, e.g., for convenience. Accordingly, the chimeric protein may or may not be xenogeneic-derived relative either to the species of cell in which the protein is expressed or the subject to which the protein is administered.

In some embodiments, the native inhibitory receptor is selected from proteins that reduce immune activity upon binding a native agonist. For example, the native inhibitory receptor may reduce T cell proliferation, T cell survival, cytokine secretion, or immune cytolytic activity upon binding a native agonist. The native inhibitory receptor may be a lymphocyte inhibitory receptor (i.e., the inhibitory receptor may be expressed on lymphocytes, such as T cells). For example, the native inhibitory receptor may be expressed on T cells, and the binding of an agonist to the native inhibitory receptor may cause cell signaling that disfavors T cell proliferation, T cell survival, cytokine secretion, or immune cytolytic activity.

In some embodiments, the native inhibitory receptor may be CTLA-4 (cytotoxic T-lymphocyte-associated protein 4; CD152), PD-1 (Programmed cell death protein 1; CD279), LAG-3 (Lymphocyte-activation gene 3; CD223), or Tim-3 (T cell immunoglobulin mucin-3). Thus, in some embodiments, the extracellular domain may be the extracellular domain from CTLA-4, PD-1, LAG-3, or Tim-3. The inhibitory receptor may be PD-1. In some embodiments, the transmembrane protein comprises the extracellular domain of PD-1. In some embodiments, the sequence of the extracellular domain comprises

(SEQ ID NO: 7)
PGWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSGT
YLCGAISLAPKAQIKESLRAELRVTERRAEVPTARPSPSPRPAGQFQTL
V..

In some embodiments, the intracellular signaling domain is the signaling domain of an intracellular signaling protein. In some embodiments, the intracellular signaling domain may comprise kinase activity or a phosphorylation site. The intracellular signaling domain can, in some embodiments, activate a signaling molecule, such as a kinase or phosphorylase, e.g., following signal transduction across a cell membrane. The intracellular signaling domain may signal through a downstream kinase or a phosphorylase.

The intracellular signaling protein may be a human protein or a mouse protein. Thus, the intracellular signaling domain may comprise a human or mouse amino acid sequence. In some embodiments, the intracellular signaling protein is selected to match the species of a subject and cell that is being used for treatment, e.g., so that the signaling domain may utilize the cell's cytosolic machinery to activate downstream signaling molecules. Nevertheless, the intracellular signaling protein may be selected from a different species, e.g., for convenience, such as described above.

In some embodiments, the intracellular signaling protein increases immune activity. Thus, signal transduction via the chimeric transmembrane protein can result in a signal cascade that increases immune activity, wherein the intracellular signaling domain mediates the intracellular signaling cascade. In some embodiments, the intracellular signaling protein can enhance T cell proliferation, T cell survival, cytokine secretion, or immune cytolytic activity. In some embodiments, the intracellular signaling protein is a transmembrane protein or the intracellular signaling protein can bind a native transmembrane protein. The intracellular signaling protein may be a lymphocyte protein (i.e., the intracellular signaling protein may be expressed on lymphocytes, such as T cells). In some embodiments, the intracellular signaling protein is CD3ζ (T-cell surface glycoprotein CD3 zeta chain; CD247), 4-1BB (tumor necrosis factor receptor superfamily member 9; CD137), or CD28 (T-cell-specific surface glycoprotein CD28; Tp44). Thus, the intracellular signaling protein may comprise a signaling domain from CD3ζ, 4-1BB, or CD28. The intracellular signaling protein may be 4-1BB. Thus, the intracellular signaling protein may comprise a signaling domain from 4-1BB. In some embodiments, the intracellular domain comprises

(SEQ ID NO: 8)
KRGRKKLLYIFKQPFMRPVQTTQEEDGCSCRFPEEEEGGCEL.

In some embodiments, the chimeric transmembrane protein comprises a suicide domain, i.e., to kill a recombinant cell comprising the protein. The suicide domain may comprise thymidine kinase activity or caspase activity. For example, the suicide domain may be a thymidine kinase or a caspase. In some embodiments, the suicide domain is the thymidine kinase domain of HSV thymidine kinase (“HSV-TK”) or the suicide domain comprises a portion of caspase 9.

In some aspects, the embodiments relates to a nucleic acid molecule encoding a chimeric transmembrane protein as described herein. The nucleic acid molecule may comprise a promoter, wherein the promoter is operably linked to a nucleotide sequence encoding the chimeric transmembrane protein, e.g., for expression of a chimeric transmembrane protein in a recombinant cell. In some embodiments, the promoter is a constitutive promoter. In some embodiments, the promoter is a cell specific promoter. In some embodiments, the promoter is a tissue specific promoter.

The nucleic acid molecule may comprise the sequence set forth in SEQ ID NO:1, SEQ ID NO:2, SEQ ID NO:3, or SEQ ID NO:4. The nucleic acid molecule may comprise at least about 100, 200, 300, 400, 500, 600, or 700 consecutive nucleotides in the sequence set forth in SEQ ID NO:1, SEQ ID NO:2, SEQ ID NO:3, or SEQ ID NO:4. The nucleic acid molecule may comprise a nucleotide sequence having at least about 90%, 91%, 92%, 93%, 94%, 95%, 96%, 97%, 98%, or 99% sequence homology with the nucleotide sequence set forth in SEQ ID NO:1, SEQ ID NO:2, SEQ ID NO:3, or SEQ ID NO:4. The nucleic acid molecule may comprise a nucleotide sequence having at least about 90%, 91%, 92%, 93%, 94%, 95%, 96%, 97%, 98%, or 99% sequence homology with at least about 100, 200, 300, 400, 500, 600, or 700 consecutive nucleotides in the nucleotide sequence set forth in SEQ ID NO:1, SEQ ID NO:2, SEQ ID NO:3, or SEQ ID NO:4. For example, the nucleic acid molecule may comprise a nucleotide sequence having at least 95% sequence homology with at least 100 consecutive nucleotides in the nucleotide sequence set forth in SEQ ID NO:3.

In some embodiments, the nucleic acid molecule encodes an amino acid sequence as described herein and/or in the drawings. In some embodiments, the nucleic acid molecule encodes an amino acid sequence comprising one or more amino acid sequences set forth in SEQ ID NO: 5, SEQ ID NO: 6, SEQ ID NO: 7, SEQ ID NO: 8, SEQ ID NO: 9, SEQ ID NO: 10, or SEQ ID NO: 11. In some embodiments, the nucleic acid molecule may comprise a nucleotide sequence that encodes an amino acid sequence having at least about 90%, 91%, 92%, 93%, 94%, 95%, 96%, 97%, 98%, or 99% sequence homology with a nucleotide sequence set forth herein and/or in the drawings. Homology can be identity or similarly in the context of a protein. Sequence homology may refer to sequence identity in the context of a nucleic acid molecule. Homology can be used by employing routine tools such as Expasy, BLASTp, Clustal, and the like using default settings.

In some embodiments, the chimeric transmembrane protein comprises one or more amino acid sequences set forth in the following table:

| Sequence | SEQ ID NO |
|---|---|
| IISFFLALTSTALLFLLFFLTLRFSVV | 5 |
| MALPVTALLLPLALLLHAARP | 6 |
| PGWFLDSPDRPWNPPTFSPALLVVTEGDNATFTC | 7 |
| SFSNTSESFVLNWYRMSPSNQTDKLAAFPEDRSQ | |
| PGQDCRFRVTQLPNGRDFHMSVVRARRNDSGTY | |
| LCGAISLAPKAQIKESLRAELRVTERRAEVPTAHP | |
| SPSPRPAGQFQTLV | |
| KRGRKKLLYIFKQPFMRPVQTTQEEDGCSCRFPE | 8 |
| EEEGGCEL | |
| IISFFLALTSTALLFLLFFLTLRFSVVKRGRKKLLY | 9 |
| IFKQPFMRPVQTTQEEDGCSCRFPEEEEGGCEL | |
| PGWFLDSPDRPWNPPTFSPALLVVTEGDNATFTC | 10 |
| SFSNTSESFVLNWYRMSPSNQTDKLAAFPEDRSQ | |
| PGQDCRFRVTQLPNGRDFHMSVVRARRNDSGTY | |
| LCGAISLAPKAQIKESLRAELRVTERRAEVPTAHP | |
| SPSPRPAGQFQTLVIISFFLALTSTALLFLLFFLTLR | |
| FSVVKRGRKKLLYIFKQPFMRPVQTTQEEDGCSC | |
| RFPEEEEGGCEL | |
| MALPVTALLLPLALLLHAARPPGWFLDSPDRPW | 11 |
| NPPTFSPALLVVTEGDNATFTCSFSNTSESFVLNW | |
| YRMSPSNQTDKLAAFPEDRSQPGQDCRFRVTQLP | |
| NGRDFHMSVVRARRNDSGTYLCGAISLAPKAQI | |
| KESLRAELRVTERRAEVPTAHPSPSPRPAGQFQTL | |
| VIISFFLALTSTALLFLLFFLTLRFSVVKRGRKKLL | |
| GCSCRFPEEEEGGCEL | |

In some embodiments, the chimeric transmembrane protein comprises an amino acid sequence having at least about 90%, 91%, 92%, 93%, 94%, 95%, 96%, 97%, 98%, or 99% sequence homology with one of the amino acid sequences set forth herein.

Variants of the amino acid sequences described herein may be included in various embodiments. The term “variant” refers to a protein or polypeptide in which one or more (e.g., 1, 2, 3, 4, etc.) amino acid substitutions, deletions, and/or insertions are present as compared to the amino acid sequence of a protein or polypeptide, and the term includes naturally occurring allelic variants and alternative splice variants of a protein or polypeptide. The term “variant” includes the replacement of one or more amino acids in an amino acid sequence with a similar or homologous amino acid(s) or a dissimilar amino acid(s). Some variants include alanine substitutions at one or more amino acid positions in an amino acid sequence. Other substitutions include conservative substitutions that have little or no effect on the overall net charge, polarity, or hydrophobicity of the protein. Conservative substitutions may have insignificant effect on the function of the chimeric transmembrane protein. In some embodiments, the function can be the specificity of a protein when expressed in a lymphocyte, e.g., a marrow-infiltrating lymphocyte (MIL), such as described in Example 3. One of skill in the art can determine if a substitution affects the function of a chimeric transmembrane protein by comparing to the sequences provided herein using a protocol identical to, or analogous to, Example 3. Non-limiting exemplary conservative substitutions are set forth in the table below. According to some embodiments, a chimeric transmembrane protein has at least 90%, 91%, 92%, 93%, 94%, 95%, 96%, 97%, 98%, or 99% sequence identity with an amino acid sequence described herein.

### Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | proline |
| | methionine |
| | leucine |
| | isoleucine |

The table below sets out another scheme of conservative amino acid substitutions.

| Original Residue | Conservative Substitutions |
|---|---|
| Ala | Gly; Ser; Thr |
| Arg | Lys; Gln |
| Asn | Gln; His; Ser |
| Asp | Glu; Asn |
| Cys | Ser |
| Gln | Asn; Ser; Asp; Glu |
| Glu | Asp; Gln; Lys |
| Gly | Ala; Pro; Asn |
| His | Asn; Gln; Tyr |
| Ile | Leu; Val; Met; Val; Phe |
| Leu | Ile; Val; Met; Phe |
| Lys | Arg; Gln |
| Met | Leu; Tyr; Ile; Val; Phe |
| Pro | Ser; Thr; Ala; Gly |
| Phe | Met; Leu; Tyr; Trp |
| Ser | Thr; Gly; Asn; Asp |
| Thr | Ser; Asn |
| Trp | Tyr; Phe |
| Tyr | Trp; Phe |
| Val | Ile; Leu; Met; Phe |

Accordingly, in some embodiments, 1, 2, 3, 4, 5, 6, 7, 8, 9, or 10 of the amino acid residues of an amino acid sequence disclosed herein are modified with conservative substitutions. In some embodiments, only 1, 2, 3, 4 or 5 amino acid residues are substituted with conservative substitutions.

In some embodiments, the chimeric transmembrane protein comprises a sequence of SEQ ID NO: 10 or SEQ ID NO: 11 or a variant thereof. SEQ ID NO: 10 is a combination of SEQ ID NO: 5, 7, and 8. SEQ ID NO: 11 is a combination of SEQ ID NO: 5, 6, 7, and 8. In some embodiments, the sequence of SEQ ID NO: 6 is replaced with another signal peptide or leader sequence, that can assist in trafficking the chimeric transmembrane protein to the extracellular membrane. In some embodiments, the transmembrane domain, e.g., SEQ ID NO: 5, is replaced with a different transmembrane protein. In some embodiments, the transmembrane domain is the transmembrane domain of PD-1. In some embodiments, the transmembrane domain is the transmembrane domain of 4-1BB.

In some aspects, the embodiments relate to a recombinant cell, comprising a nucleic acid as disclosed herein. In some embodiments, the embodiments relate to a recombinant cell, comprising a chimeric transmembrane protein as described herein. In some embodiments, the cell comprises a chimeric protein comprising a protein of SEQ ID NO: 5, 6, 7, 8, 9, 10, or 11 or a variant thereof. In some embodiments, the cell is a lymphocyte. The cell may be a T cell. In some embodiments, the cell may be a tumor-infiltrating lymphocyte (“TIL”) or a marrow infiltrating lymphocyte (“MIL”).

In some embodiments, the cell comprising a chimeric transmembrane protein described herein persist longer and/or remain in an active state longer in a subject when administered to the subject as compared to a cell without a chimeric transmembrane protein.

In some aspects, the embodiments relate to a method for making a recombinant cell, comprising transfecting a cell with a nucleic acid molecule as described herein. In some aspects, the embodiments relate to a method for making a recombinant cell, comprising transfecting a cell with a nucleic acid molecule encoding an amino acid sequence as described herein. The nucleic acid molecule may be a plasmid. The cell can be transfected by a plasmid comprising one or more nucleotide sequences as described herein. The cell can also be infected with a virus or virus-like particle comprising the nucleic acid molecule. In some embodiments, the cell is a TIL or a MIL. In some embodiments, the MIL is an activated MIL. MILs can be activated, for example, by incubating them with anti-CD3/anti-CD28 beads and appropriate cytokines, e.g., under hypoxic conditions. An example of growing the MILs under hypoxic conditions can found, for example, in WO2016037054, which is hereby incorporated by reference in its entirety. In some embodiments, the nucleic acid molecule is transfected into a cell after the cell has been incubated in a hypoxic environment as described herein. In some embodiments, the nucleic acid molecule is transfected into a cell after the cell has been incubated in a hypoxic environment for about 1, 2, 3, 4, or 5 days. In some embodiments, the cell is then incubated under normoxic conditions for about 1, 2, 3, 4, or 5 days.

In some embodiments, a MIL comprising the chimeric transmembrane protein is prepared according to a method described in WO2016037054, which is hereby incorporated by reference in its entirety. In some embodiments, the method may comprise removing cells in the bone marrow, lymphocytes, and/or marrow infiltrating lymphocytes (“MILs”) from the subject; incubating the cells in a hypoxic environment, thereby producing activated MILs; and administering the activated MILs to the subject. The cells can also be activated in the presence of anti-CD3/anti-CD28 antibodies and cytokines as described herein. A nucleic acid molecule encoding a chimeric transmembrane protein, such as one of those described herein, can be transfected or infected into a cell before or after the MIL is incubated in a hypoxic environment.

The hypoxic environment may comprise less than about 21% oxygen, such as less than about 20%, 19%, 18%, 17%, 16%, 15%, 14%, 13%, 12%, 11%, 10%, 9%, 8%, 7%, 6%, 5%, 4%, or less than about 3% oxygen. For example, the hypoxic environment may comprise about 0% oxygen to about 20% oxygen, such as about 0% oxygen to about 19% oxygen, about 0% oxygen to about 18% oxygen, about 0% oxygen to about 17% oxygen, about 0% oxygen to about 16% oxygen, about 0% oxygen to about 15% oxygen, about 0% oxygen to about 14% oxygen, about 0% oxygen to about 13% oxygen, about 0% oxygen to about 12% oxygen, about 0% oxygen to about 11% oxygen, about 0% oxygen to about 10% oxygen, about 0% oxygen to about 9% oxygen, about 0% oxygen to about 8% oxygen, about 0% oxygen to about 7% oxygen, about 0% oxygen to about 6% oxygen, about 0% oxygen to about 5% oxygen, about 0% oxygen to about 4% oxygen, or about 0% oxygen to about 3% oxygen. In some embodiments, the hypoxic environment comprises about 1% to about 7% oxygen. In some embodiments, the hypoxic environment is about 1% to about 2% oxygen. In some embodiments, the hypoxic environment is about 0.5% to about 1.5% oxygen. In some embodiments, the hypoxic environment is about 0.5% to about 2% oxygen. The hypoxic environment may comprise about 20%, 19%, 18%, 17%, 16%, 15%, 14%, 13%, 12%, 11%, 10%, 9%, 8%, 7%, 6%, 5%, 4%, 3%, 2%, 1%, or about 0% oxygen. In some embodiments, the hypoxic environment comprises about 7%, 6%, 5%, 4%, 3%, 2%, or 1% oxygen.

Incubating MILs in a hypoxic environment may comprise incubating the MILs, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may comprise incubating the MILs for about 1 hour to about 30 days, such as about 1 day to about 20 days, about 1 day to about 14 days, or about 1 day to about 12 days. In some embodiments, incubating MILs in a hypoxic environment comprises incubating the MILs in a hypoxic environment for about 2 days to about 5 days. The method may comprise incubating MILs in a hypoxic environment for about 1 day, 2 days, 3 days, 4 days, 5 days, 6 days, 7 days, 8 day, 9 days, 10 days, 11 days, 12 days, 13 days, or 14 days. In some embodiments, the method comprises incubating the MILs in a hypoxic environment for about 3 days. In some embodiments, the method comprises incubating the MILs in a hypoxic environment for about 2 days to about 4 days. In some embodiments, the method comprises incubating the MILs in a hypoxic environment for about 3 days to about 4 days.

In some embodiments, the method further comprises incubating the MILs in a normoxic environment, e.g., after incubating the MILs in a hypoxic environment.

The normoxic environment may comprise at least about 21% oxygen. The normoxic environment may comprise about 5% oxygen to about 30% oxygen, such as about 10% oxygen to about 30% oxygen, about 15% oxygen to about 25% oxygen, about 18% oxygen to about 24% oxygen, about 19% oxygen to about 23% oxygen, or about 20% oxygen to about 22% oxygen. In some embodiments, the normoxic environment comprises about 21% oxygen.

Incubating MILs in a normoxic environment may comprise incubating the MILs, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may comprise incubating the MILs for about 1 hour to about 30 days, such as about 1 day to about 20 days, about 1 day to about 14 days, about 1 day to about 12 days, or about 2 days to about 12 days.

In some embodiments, the cell is transfected or infected with a nucleic acid molecule encoding a chimeric transmembrane protein described herein after being placed in a normoxic environment or before it is placed in a normoxic environment.

In some embodiments, the MILs are obtained by extracting a bone marrow sample from a subject and culturing/incubating the cells as described herein. In some embodiments, the bone marrow sample is centrifuged to remove red blood cells. In some embodiments, the bone marrow sample is not subject to apheresis. In some embodiments, the bone marrow sample does not comprise peripheral blood lymphocytes (“PBL”) or the bone marrow sample is substantially free of PBLs. These methods select for cells that are not the same as what have become to be known as TILs. Thus, a MIL is not a TIL. TILs can be selected by known methods to one of skill in the art and can be transfected or infected with the nucleic acid molecules described herein such that the TILs can express the chimeric transmembrane protein described herein.

In some embodiments, the cells are also activated by culturing with antibodies to CD3 and CD28. This can be performed, for example by incubating the cells with anti-CD3/anti-CD28 beads that are commercially available or that can be made by one of skill in the art. The cells can then be plated in a plate, flask, or bag. Hypoxic conditions can be achieved by flushing either the hypoxic chamber or cell culture bag for 3 minutes with a 95% Nitrogen and 5% CO₂ gas mixture. This can lead to, for example, 1-2% or less O₂ gas in the receptacle. Cells can be then cultured as described herein or as in the examples of WO2016037054, which is hereby incorporated by reference.

In some embodiments, a hypoxic MIL comprising a chimeric transmembrane protein as described herein is provided. In some embodiments, the hypoxic MIL is in an environment of about 0.5% to about 5% oxygen gas. In some embodiments, the hypoxic MIL is in an environment of about 1% to about 2% oxygen gas. In some embodiments, the hypoxic MIL is in an environment of about 1% to about 3% oxygen gas. In some embodiments, the hypoxic MIL is in an environment of about 1% to about 4% oxygen gas. A hypoxic MIL is a MIL that has been incubated in a hypoxic environment, such as those described herein, for a period of time, such as those described herein. Without being bound to any particular theory, a hypoxic MIL will undergo changes in protein and/or gene expression that affect the anti-tumor capabilities of the MIL. As described herein, the hypoxic MIL can also be activated with the presence of anti-CD3/anti-CD28 beads or other similar activating reagents. Thus, a hypoxic MIL can also be an activated-hypoxic MIL.

In some aspects, the embodiments relates to a method for increasing an immune response in a subject, comprising administering to the subject a recombinant cell as described herein. In some embodiments, the embodiments relate to a method for treating a neoplasm in a subject, comprising administering to the subject a recombinant cell as described herein. The neoplasm may be a benign neoplasm, a malignant neoplasm, or a secondary neoplasm. The neoplasm may be cancer. The neoplasm may be a lymphoma or a leukemia, such as chronic lymphocytic leukemia (“CLL”) or acute lymphoblastic leukemia (“ALL”). The neoplasm may be multiple myeloma as well as any solid tumor (e.g., breast cancer, prostate cancer, lung cancer, esophageal cancer, brain cancer, kidney cancer, bladder cancer, pancreatic cancer, osteosarcoma, and the like).

The method may comprise administering to the subject a plurality of recombinant cells as described herein. The method may comprise administering to the subject an effective amount of recombinant cells as described herein.

In some embodiments, the cell is obtained from the subject. The cell that is transfected or infected may be obtained from the subject. The cell can be obtained as described herein. For example, a cell that is administered may be autologous to the subject. In some embodiments, the cell that is administered is allogeneic to the subject. The cell may be obtained from the subject and transfected or infected with a nucleic acid encoding a chimeric transmembrane protein as described herein. The cell may be a daughter cell, wherein a parent of the daughter cell was obtained from the subject. The recombinant cell may have been transfected or infected with the nucleic acid or a parent of the recombinant cell may have been transfected or infected with the nucleic acid. In some embodiments, the cell after being transfected or infected expresses a protein comprising one or more of the amino sequences described herein.

The method may further comprise making the recombinant cell, wherein making the recombinant cell comprises transfecting or infecting a cell with a nucleic acid encoding a chimeric transmembrane protein, such as those described herein. In some embodiments, the chimeric transmembrane protein comprises an amino acid sequence set forth in any one of SEQ ID NO: 5, 6, 7, 8, 9, 10, or 11 or a variant thereof. Similarly, the method may further comprise making a plurality of recombinant cells, wherein making the plurality of recombinant cells comprises transfecting or infecting a plurality of cells with nucleic acids encoding a chimeric transmembrane protein, such as those described herein. The method may further comprise expanding a parent cell, e.g., the recombinant cell may be a daughter cell of the parent cell. The method may comprise expanding a population of cells, e.g., the method may comprise administering to the subject a plurality of recombinant cells as described herein, and each cell of the plurality of recombinant cells may be a daughter cell of a parent cell.

The method may further comprise isolating the cell or a parent cell from the subject.

The method may further comprise sorting the cell, e.g., by fluorescence activated cell sorting (“FACS”) or magnetic activated cell sorting (“MACS”).

The cells can be administered to a subject by any suitable route in, for example, a pharmaceutically acceptable composition. In some embodiments, the composition is pyrogen free. For example, administration of the cells may be carried out using any method known in the art. For example, administration may be parenteral, intravenous, intra-arterial, subcutaneous, intramuscular, intracranial, intraorbital, ophthalmic, intraventricular, intracapsular, intraspinal, intracisternal, intraperitoneal, intracerebroventricular, or intrathecal. For parenteral administration, the cells may be administered by either intravenous, subcutaneous, or intramuscular injection, in compositions with pharmaceutically acceptable vehicles or carriers. The cells can be formulated for parenteral administration by injection, for example, by bolus injection or continuous infusion. The compositions can take such forms as suspensions, solutions, or emulsions in oily or aqueous vehicles, and can contain formulatory agents, for example, suspending, stabilizing, and/or dispersing agents.

For administration by injection, it can be desired to use the cells in solution in a sterile aqueous vehicle which may also contain other solutes such as buffers or preservatives as well as sufficient quantities of pharmaceutically acceptable salts or of glucose to make the solution isotonic. In some embodiments, the pharmaceutical compositions may be formulated with a pharmaceutically acceptable carrier to provide sterile solutions or suspensions for injectable administration. In particular, injectables can be prepared in conventional forms, either as liquid solutions or suspensions or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. Suitable pharmaceutical carriers are described in “Remington's pharmaceutical Sciences” by E. W. Martin.

The subject may be any organism that comprises immune cells. For example, the subject may be selected from rodents, canines, felines, porcines, ovines, bovines, equines, and primates. The subject may be a mouse or a human.

The subject may have a neoplasm. The neoplasm may be a benign neoplasm, a malignant neoplasm, or a secondary neoplasm. The neoplasm may be cancer. The neoplasm may be a lymphoma or a leukemia, such as chronic lymphocytic leukemia (“CLL”) or acute lymphoblastic leukemia (“ALL”). The subject may have a glioblastoma, medulloblastoma, breast cancer, head and neck cancer, kidney cancer, ovarian cancer, Kaposi's sarcoma, acute myelogenous leukemia, and B-lineage malignancies. The subject may have multiple myeloma.

In some embodiments, the subject is a subject “in need thereof” As used herein, the phrase “in need thereof” means that the subject has been identified or suspected as having a need for the particular method or treatment. In some embodiments, the identification can be by any means of diagnosis. In any of the methods and treatments described herein, the subject can be in need thereof.

As used herein, terms such as “a,” “an,” and “the” include singular and plural referents unless the context clearly demands otherwise.

As used in this document, terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used herein, the terms “treat,” “treated,” or “treating” mean both therapeutic treatment wherein the object is to slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of cancer” or “treating cancer” means an activity that alleviates or ameliorates any of the primary phenomena or secondary symptoms associated with the cancer or any other condition described herein. In some embodiments, the cancer that is being treated is one of the cancers recited herein.

### EXAMPLES

The following examples are illustrative, but not limiting, of the methods and compositions described herein. Other suitable modifications and adaptations of the variety of conditions and parameters normally encountered in therapy and that are obvious to those skilled in the art are within the spirit and scope of the embodiments.

### Example 1: CAR Transduction Protocol

16-24 hours prior to transduction, T-cells were plated in an appropriate media and were stimulated with CD3, CD28 and IL-2. The cells were then placed in an incubator (37° C./5% CO₂) overnight. After 16-24 hours, as much media as possible was removed without disturbing the cells. The CAR virus was then added to the cells and placed back in the incubator for 4-12 hours. After 4-12 hours, the appropriate volume of media containing IL-2 was added back to the cells and then placed back in the incubator. Cells were left in the incubator to grow, splitting and changing media when necessary, for 3-12 days. CAR transduction may be checked by a variety of methods including, but not limited to flow cytometry, western blotting or fluorescence microscopy, if a fluorescent reporter gene has been used.

### Example 2: CAR Transfection Protocol

293T cells were passaged every two days in DMEM+10% FBS for at least three passages at a cell density at which they never became more than 80% confluent. One day prior to transfection, the 293T cells were seeded at a density at which they were about 80% confluent after 24 hours (on the day of transfection). On the day of transfection, media was removed and enough fresh media was added to cover the cells. In a separate tube, VSV-G, Gag, Pol & Rev plasmids, a transfection reagent and the CAR plasmid were combined and incubated at room temperature for 10-20 minutes. This mixture was then added drop-wise to the 293T cells and incubated overnight. 12-24 hours after transfection, the media was either completely changed or additional fresh media was added. At both 48 hrs and 72 hrs post-transfection, virus-containing media from the cells was collected and cells were replenished with fresh media. Any cells in the collected media were removed by centrifugation or filtration. The collected media was then spun in an ultracentrifuge to pellet the virus. Excess media was removed and the virus was re-suspended in DMEM or HBSS, aliquoted into sterile tubes and stored at −80° C. until used.

### Example 3: Mil Function and Growth is not Negatively Affected by the Presence of a Chimeric Receptor Protein

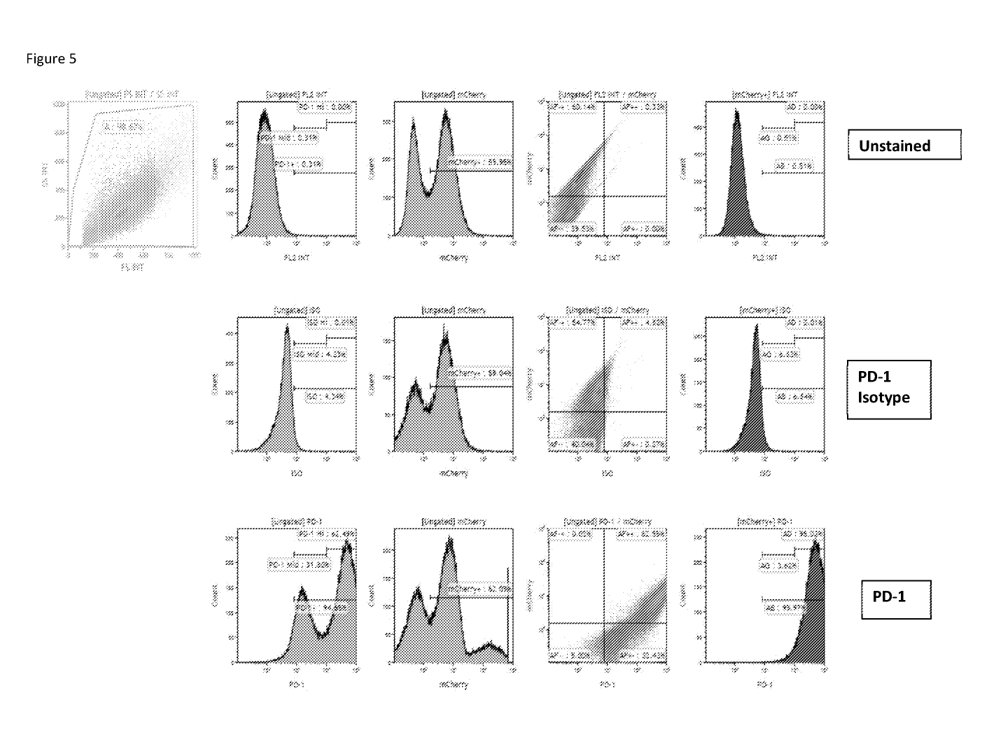

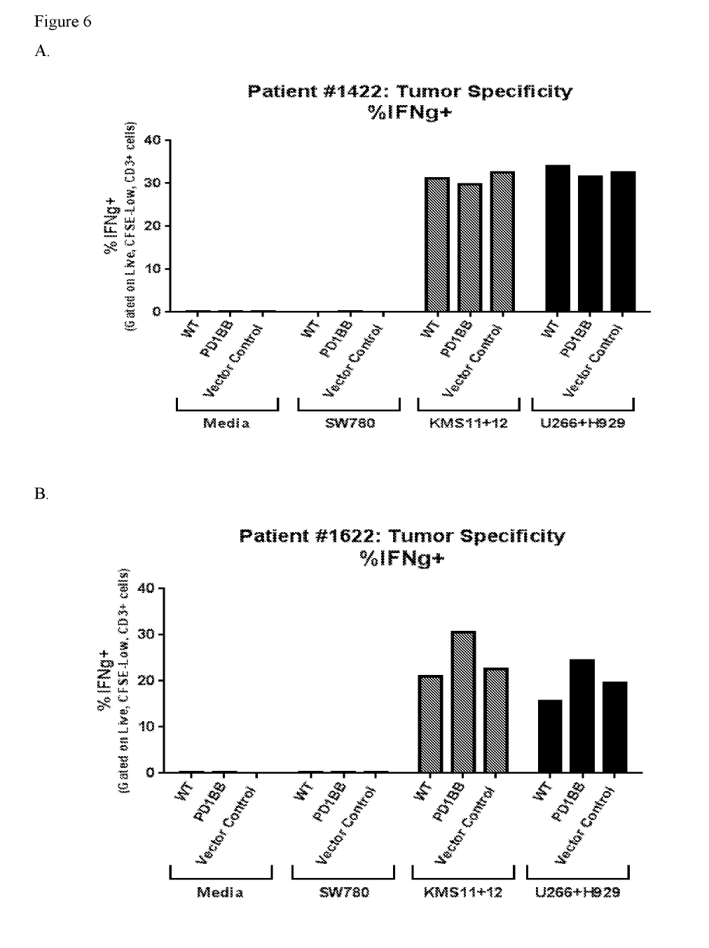

MILs obtained from subjects were activated and expanded as described herein. Briefly, after the marrow sample was obtained from the subject, the cells were incubated under hypoxic conditions in the presence of anti-CD3/anti-CD28 beads and cytokines as described in WO2016037054, which is hereby incorporated by reference. The MILs were then infected with a virus comprising a nucleic acid molecule encoding a chimeric transmembrane protein comprising SEQ ID NO: 11. The cells were then grown under normoxic conditions and allowed to expand. The control and infected MILs were contacted with different cell types. Neither the expansion of the MILS nor the ability of the MILs to recognize antigens was negatively affected by the presence of the chimeric transmembrane protein. These results demonstrate that adding a chimeric transmembrane protein to a MIL is not detrimental to its functions and growth. The results are illustrated in FIG. 6, Panel A and B, which are from two different patients.

In summary, the embodiments and examples provided herein demonstrate that cells expressing a chimeric transmembrane protein can be effectively used to treat cancer and/or modulate an immune response.

Any U.S. patents, U.S. patent application publications, U.S. patent applications, foreign patents, foreign patent applications and non-patent publications, including CAS numbers, referred to in this specification and/or listed in the Application Data Sheet are incorporated herein by reference, in their entirety.

What is claimed is:

1. A chimeric transmembrane protein, comprising:
the extracellular domain of an inhibitory receptor; and
an intracellular signaling domain that can activate an immune response, wherein the intracellular signaling domain comprises a portion of an intracellular signaling protein.

2. The protein of claim 1, wherein the protein comprises a sequence selected from the group consisting of SEQ ID NOs: 7-10.

3-5. (canceled)

6. The protein of claim 1, wherein the intracellular signaling domain comprises kinase activity.

7. The protein of claim 1, wherein the intracellular signaling domain comprises a phosphorylation site.

8. The protein of claim 1, wherein the protein comprises the transmembrane domain of the inhibitory receptor or the transmembrane domain of the intracellular signaling protein.

9. (canceled)

10. The protein of claim 1, wherein the inhibitory receptor reduces immune activity upon binding a native agonist.

11. The protein of claim 1, wherein the inhibitory receptor can reduce T cell proliferation, T cell survival, cytokine secretion, or immune cytolytic activity upon binding a native agonist.

12. The protein of claim 1, wherein the inhibitory receptor is a lymphocyte inhibitory receptor.

13. The protein of claim 12, wherein the inhibitory receptor is CTLA-4, PD-1, LAG-3, or Tim-3.

14. (canceled)

15. (canceled)

16. The protein of claim 1, wherein the intracellular signaling protein increases immune activity.

17. The protein of claim 1, wherein the intracellular signaling protein can enhance T cell proliferation, T cell survival, cytokine secretion, or immune cytolytic activity.

18. The protein of claim 1, wherein the intracellular signaling protein is a transmembrane protein or the intracellular signaling protein can bind a native transmembrane protein.

19. The protein of claim 1, wherein the intracellular signaling protein is a lymphocyte protein.

20. The protein of claim 19, wherein the intracellular signaling protein is CD3ζ, 4-1BB, or CD28.

21. (canceled)

22. The protein of claim 1, further comprising a suicide domain.

23. The protein of claim 22, wherein the suicide domain has thymidine kinase activity or the suicide domain is a caspase.

24. (canceled)

25. A nucleic acid encoding the chimeric transmembrane protein of claim 1.

26. (canceled)

27. A recombinant cell, comprising the chimeric transmembrane protein of claim 1.

28. The cell of 27, wherein the cell is a T cell, a tumor infiltrating lymphocyte (“TIL”), a marrow infiltrating lymphocyte (“MIL”), or a lymphocyte.

29-39. (canceled)

40. A method for increasing an immune response or treating a neoplasm in a subject, comprising administering to the subject the recombinant cell of claim 27.

41-54. (canceled)
